(19) United States
(12) Patent Application Publication
(10) Pub. No.: US 2025/0352975 A1
(43) Pub. Date: Nov. 20, 2025

(54) SHELL-AND-TUBE HEAT EXCHANGE REACTOR FOR CARRYING OUT A CATALYTIC GAS-PHASE PARTIAL OXIDATION REACTION AND PROCESS FOR CARRYING OUT A CATALYTIC GAS-PHASE PARTIAL OXIDATION

(71) Applicant: BASF SE, Ludwigshafen am Rhein (DE)

(72) Inventors: Sven REINING, Ludwigshafen am Rhein (DE); Gerhard OLBERT, Dossenheim (DE); Ralf BOEHLING, Ludwigshafen am Rhein (DE); Jochen GAUER, Ludwigshafen am Rhein (DE); Kai Rainer EHRHARDT, Ludwigshafen am Rhein (DE)

(21) Appl. No.: 18/874,169

(22) PCT Filed: Jun. 2, 2023
(86) PCT No.: PCT/EP2023/064854

§ 371 Date: Dec. 12, 2024

(30) Foreign Application Priority Data
Jun. 14, 2022 (EP) 22178917.5

(51) Int. Cl. B01J 19/24; B01J 12/00; B01J 19/00; C07C 45/38; C07C 47/21

(52) U.S. Cl. CPC B01J 19/2415; B01J 12/007; B01J 19/0013; C07C 45/38; C07C 47/21; B01J 2219/00081; B01J 2219/00159; B01J 2219/19

(57) ABSTRACT

A shell-and-tube heat exchange reactor for carrying out a catalytic gas-phase partial oxidation reaction comprises a shell-side heat exchange passage for circulating a heat transfer medium and a reaction passage comprising a plurality of reaction tubes; an inlet for introducing the reactant stream to the reaction passage; and an outlet from the reaction passage for recovering an effluent stream from the reaction tubes. The reaction tubes comprise a reactant pre-heating zone adjacent to the inlet, and a reaction zone downstream of the reactant pre-heating zone, the reaction zone having a catalytically active wire matrix insert having at least on a part of its surface a catalytically active precious metal. The reactor requires less frequent maintenance in the form of regeneration and/or replacement of the catalyst. The catalyst can be easily placed into the reaction tubes, and be removed therefrom. Only the portion of the entire reactant stream that travels near the hot reaction tube wall is heated up. Consequently, the portion of the reactant stream flowing in the center of the reaction tube is not heated to the reaction temperature and blind reactions of the unstable starting materials are thus reduced or even avoided.

The present invention relates to a shell-and-tube heat exchange reactor for carrying out a catalytic gas-phase partial oxidation reaction comprising a shell-side heat exchange passage for circulating a heat transfer medium and a reaction passage comprising a plurality of reaction tubes; an inlet for introducing the reactant stream to the reaction passage; and an outlet from the reaction passage for recovering an effluent stream from the reaction tubes and a process for carrying out a catalytic gas-phase partial oxidation reaction in the shell-and-tube heat exchange reactor. The invention further relates to processes for the preparation of prenol, 3,7-dimethyl-octa-2,6-dienal (citral), menthol and linalool.

Catalytic gas-phase reactions in chemical industry, such as oxidation, hydrogenation, dehydrogenation, nitration, alkylation etc., are usually performed using shell-and-tube reactors using solid-state catalysts arranged in fixed beds. Such reactions performed in shell-and-tube reactors may be either endothermic or exothermic. The fixed bed is located in reaction tubes of the shell-and-tube reactor. Nowadays, commonly used shell-and-tube reactors may have at least 5,000 and up to 45,000 reaction tubes. This plurality of reaction tubes is referred to as reaction tube bundle, which is generally annular and arranged vertically and surrounded by a reactor shell. Both ends of said reaction tube bundle are sealed in tube sheets. A feed gas stream is usually introduced at the top part of the shell-and-tube reactor via a hood and fed to the reaction tubes via the tube sheet. A resulting product gas mixture is discharged at the bottom part of the shell-and-tube reactor via the opposite tube sheet and hood. Alternatively, the feed gas stream is introduced at the bottom part of the shell-and-tube reactor and leaves the shell-and-tube reactor via the upper tube sheet and hood.

Catalysts comprising a catalytically active precious metal such as copper or silver upon a suitable support are known to be useful in catalyzing certain chemical oxidation reactions. Commonly used catalysts for fixed beds of state-of-the-art processes involve, e.g., porous solid-state catalysts impregnated with silver, e.g. for the oxidation of ethylene to ethylene oxide, or shell catalysts coated with silver, e.g. for the oxidation of primary alcohols to aldehydes, for example of isoprenol to prenal.

Introducing such catalysts which consist of individual catalyst bodies into the reaction tubes and settling the catalyst to a fixed bed is a complex and time-consuming process, as, for example, a filling level measurement is required in order to verify that a sufficient amount of catalyst is present inside the reaction tube.

Vapor-phase fixed-bed tubular catalytic reactors often exhibit an undesirable temperature profile along the reactor tube length. Typically, the temperature profile of an exothermic catalytic reaction is low at the inlet, rises to a maximum and then drops off as the reactant stream is starved of reactants.

Reactor temperatures which are outside of the optimum temperature range for a given reaction result in lower selectivity as undesirable products are formed. This results in a deposition of organic constituents of the feed gas stream on the surface of the active catalyst material in the reaction tubes in the form of carbonaceous deposits, e.g. in the form of coke, in the case of temperatures lower than the ignition temperature of the reaction. As a consequence of this deposition, a part of the active catalyst material is deactivated and also the pressure drop increases with increasing deposition. Thus, regular maintenance in the form of regeneration and/or even replacement of the catalyst is required. For example, in the oxidation of isoprenol to prenal using the abovementioned shell catalysts coated with silver, it is necessary to carry out a maintenance step in the form of regeneration once every week. As a result of that, the number of annual operating hours is significantly reduced and the existing production capacities cannot be fully utilized.

Furthermore, with catalytic materials that have high thermal conductivity, such as copper or silver, a “migrating hotspot” may occur, i.e. a hotspot which migrates in the direction of the reaction tube inlet. As a result, the residence time of the formed target product is prolonged, which in turn leads to subsequent reactions of the target product and reduces the selectivity to the target product.

The occurrence of a pronounced and undesirable temperature “hump” or “hotspot” is caused by poor heat transfer between the catalyst in the reactor tubes and the heat transfer medium. In order to mitigate the problem, it has been attempted to precede the catalyst-filled reaction area of the reaction tube by a non-catalytic heat exchange area in which the reactant stream is heated to the reaction onset temperature before it comes into contact with the catalyst. Also, in order to avoid to the extent possible further undesired reactions of the product and to recover the product in a form as unchanged as possible, it has been suggested that a heat exchange area be provided downstream of the reaction area, wherein the effluent stream is heat-exchanged with the heat transfer medium and thereby cooled.

In order to optimize the function of heat exchange areas for use on an industrial scale, various packing materials present as individual elements, such as balls or rings have been recommended as flow obstacles in the heat exchange area. These packing materials are, however, disadvantageous, since, on the one hand, they lead to significant loss of pressure and, furthermore, a rapid deposition of combustion residues due to their high specific surface area. In addition, the heat exchange via the reaction tube wall is not efficient as the reactant stream is forced through the voids in the packing of the individual elements.

US 2007/274882 relates to a reactor comprising at least: (a) a reaction area comprising at least one solid-state catalyst; and (b) a coolable heat exchanger area comprising at least one housing at least partially accommodating an insert, wherein the reaction area and the coolable heat exchanger area are in fluid-communication.

US 2012/0277473 describes a process for producing C₁-C₁₀ aldehydes by oxidative dehydrogenation of C₁-C₁₀ alcohols over a shaped catalyst body obtainable by three-dimensional shaping and/or arranging in space of silver-containing fibers and/or threads. The average diameter or the average diagonal length of an essentially rectangular or square cross section of these silver-containing fibers and/or threads is in the range from 30 μm to 200 μm.

It is therefore an object of the present invention to provide a reactor for carrying out a catalytic gas-phase partial oxidation reaction requiring less frequent maintenance in the form of regeneration and/or replacement of the catalyst. It is a further object of the invention to facilitate placing the catalyst into the reaction tubes, and removing the same therefrom.

Accordingly, the invention relates to a shell-and-tube heat exchange reactor for carrying out a catalytic gas-phase partial oxidation reaction comprising
a shell-side heat exchange passage for circulating a heat transfer medium and a reaction passage comprising a plurality of reaction tubes;
an inlet for introducing the reactant stream to the reaction passage; and
an outlet from the reaction passage for recovering an effluent stream from the reaction tubes;
   wherein the reaction tubes comprise
   a reactant pre-heating zone adjacent to the inlet, and
   a reaction zone downstream of the reactant pre-heating zone, the reaction zone having a catalytically active wire matrix insert having at least on a part of its surface a catalytically active precious metal.

Herein, “downstream” or “upstream” is with respect to a flow direction of the reactant stream.

The term “reactant pre-heating zone” denotes a section of the reaction tube, i.e. a section inside the reaction tube, where essentially no catalytic gas-phase partial oxidation reaction occurs and where the gaseous stream through the reaction tubes is heat-exchanged via the tube wall with the circulating heat transfer medium. The pre-heating zone upstream of the reaction zone involves net heat flow into the reaction tube and ensures that the reactant stream is sufficiently heated up to a temperature close to or at the reaction temperature when it reaches the reaction zone.

Upon contact with the catalytic surface, the oxidation reaction immediately starts. Otherwise, in the event when a “cold” reactant stream reaches the catalytic surface such that the reaction onset temperature of the reaction is not reached, coke formation may occur. Less coke formation advantageously leads to a prolonged reactor operation without the necessity of burning off the coke from the catalytic surface.

Preferably, the reactant pre-heating zone is adapted to allow for laminar flow of the reactant inside the reactant pre-heating zone. This means, the reactant pre-heating zone is devoid of any obstacles to the reactant flow that triggers a laminar-to-turbulent flow transition. Hence, the reactant pre-heating zone preferably has an essentially free cross section, i.e. the pre-heating zone is empty.

In the case of an “essentially free cross section”, the reactant pre-heating zone may be empty. Alternatively, the reactant pre-heating zone may accommodate fixtures made of a material having zero or limited catalytic activity, which fixtures have a negligible cross-section in a plane perpendicular to the longitudinal axis of the reaction tube. Said fixtures may be attached to the catalytically active wire matrix which is present in the reaction zone and allow to easily place said wire-matrix insert into or remove the same from the reaction zone. For example, the negligible mounting may be a stainless steel wire or rod.

This setup allows for heating up only the portion of the entire reactant stream that travels near the hot reaction tube wall. Consequently, the portion of the reactant stream flowing in the center of the reaction tube is not heated to the reaction temperature and blind reactions of the unstable starting materials are thus reduced or even avoided. A “blind reaction” is an unselective oxidative reaction that occurs in the absence of the catalyst. Once the reactant stream reaches the reaction zone, the oxidation reaction is initiated. Due to the exothermic nature of this reaction, energy is released and the remainder of the reactant stream is rapidly heated to the reaction onset temperature, and the reaction proceeds. This fast heat up of the predominant part of the reaction mixture reduces unwanted side-reactions and thus leads to an increased selectivity.

Alternatively, the reactant pre-heating zone may have a wire matrix insert having zero or limited catalytic activity. The wire matrix insert may reduce or eliminate temperature gradients without creating any obstruction to flow that would promote turbulent flow characteristics. A wire matrix insert is considered as having zero catalytic activity (or in other words, as being “inert”) if it does not catalyze the gas-phase partial oxidation reaction in question to a significant degree, and the chemical composition of a stream passing the wire matrix insert does not change significantly. Similarly, a matrix insert is considered as having limited catalytic activity if its catalytic activity is less than the activity of a reaction zone. In an embodiment, the wire matrix insert having zero or limited catalytic activity is made of an inert material, preferably stainless steel.

Herein, the term “reaction zone” denotes a region of the reaction tube where the catalytic gas-phase partial oxidation reaction occurs. According to the invention, the reaction zone comprises a catalytically active wire matrix insert having at least on a part of its surface a catalytically active precious metal. Due to the more open structure of the wire matrix contained in the reaction zone as compared to a packing of individual elements, a larger proportion of the reaction heat is discharged to the reaction tube wall by radiation and does not have to be dissipated by the reactant stream. Due the unique flow characteristic of the reactant stream through the reaction tube with the wire matrix insert in place, heat transfer via the tube wall is improved. Formation of prominent hotspots can be avoided. This in turn, avoids deposition of organic constituents of the reactant stream on the surface of the active catalyst material with concomitant pressure drop. Overall, less regular maintenance in the form of regeneration and/or replacement of the catalyst is required. The number of annual operating hours can be increased and the existing production capacities can be fully utilized, reducing operation cost and increasing profit.

In contrast to individually present catalyst bodies, the wire matrix inserts can be formed contiguously, or in one piece. Hence, placing the wire matrix inserts in the catalyst containment region of the reaction tubes, and removal therefrom is much facilitated.

The “reaction zone” may be comprised of a single contiguous reaction zone. Alternatively, the reaction zone may comprise an alternating series of regions having catalytically active wire matrix inserts and regions having an essentially free cross section or having wire matrix inserts having zero or limited catalytic activity.

A “wire matrix insert” is understood to be a self-supporting skeletal-like structure made of coiled, bent or crimped metal wire which is adapted to be inserted into a reaction tube of a shell-and-tube reactor. The wire matrix insert has a more voluminous structure than a longitudinal wire.

A fixture such as a stainless steel wire or rod may be attached to the wire matrix insert which allows for easily placing the wire-matrix insert into or removing the same from the reaction zone.

In an embodiment, the catalytically active wire matrix inserts comprise an elongated core having a plurality of wire loops extending from the elongated core, wherein the wire loops are longitudinally arranged and helically shifted, that is, neighboring wire loops have an angular offset. The loops may be formed by helically bending the wire over the length of the wire matrix insert. In view of the ease of manufacture, the elongated core preferably comprises at least two longitudinal core wire members, which are twisted around each other to form core wire windings, and the wire loops are accommodated in the core wire windings.

The wire loops may be formed from one wire, or more than one intertwined wires, preferably 4 intertwined wires.

The wire matrix insert comprised in the reaction zone has at least on a part of its surface a catalytically active precious metal. The wire constituting the wire loops may be a massive precious metal wire, or a wire coated with a precious metal. The core wire may be made of brass alloys, or high-grade steels. The coating layer of precious metal superimposed on the surface of the core has a thickness of, e.g., 10 μm. In general however, a massive precious metal wire has better service life and is preferred. If the wire loops are formed from more than one intertwined wires, at least one of the intertwined wires is made of a massive precious metal wire, or a wire coated with a precious metal while the other intertwined wires can be made of an inert material.

Generally, the catalytically active precious metal is selected from copper, silver, palladium, platinum, ruthenium, and rhodium, preferably silver. A silver wire which is of the same composition throughout its cross section and comprises at least 92.5 wt.-% Ag can suitably be used. The silver wire is helically bent to form wire loops, and combined with at least two longitudinal core wire members, which are twisted around each other to form core wire windings, and the wire loops are accommodated in the core wire windings. The longitudinal core wire members can also be silver wire or inert metal wire.

Generally, the catalytically active wire matrix inserts have a cylindrical enveloping surface with a diameter matching with the inner diameter of the reaction tubes. This includes a situation where the diameter of the cylindrical enveloping surface of the undeployed wire matrix insert is slightly larger than the inner diameter of the reaction tubes. Due to the springy or elastic nature of the wire matrix insert, it can be inserted into the reaction tubes with a slight counter pressure such that the wire loops fit tightly against the inner walls of the reaction tube.

Suitable structures of wire matrix inserts are known as such. GB 2 097910 Some inserts of this type are disclosed in GB patent 1 570 530. Other inserts, as well as processes for their production are disclosed in GB 2 097 910 A. Matrix inserts are commercially available from the company Cal Gavin Ltd., England, and sold under the trade name HiTRAN®.

In an embodiment, the reaction zone of the reaction tubes has a void fraction of 0.60 to 0.99, preferably 0.80 to 0.97, more preferably 0.89 to 0.94. “Void fraction” is defined as the ratio of the void volume (i.e. the total volume of void spaces within a cylinder that envelopes the wire matrix insert) to the total volume occupied by the wire matrix insert (i.e. the volume of the cylinder that envelopes the wire matrix insert). Put otherwise, the void fraction is defined as the ratio of the void volume to the sum of the void volume plus the volume occupied by the wire loops and the elongated core constituting the wire matrix insert.

Due to the higher void fraction of the wire matrix contained in the reaction zone as compared to a packing of individual elements, such as a fixed bed of individual catalyst particles, a larger proportion of the reaction heat is discharged to the reaction tube wall by radiation and does not have to be dissipated by the reactant stream.

In an embodiment, the catalytically active wire matrix insert is adapted to enable radial mixing of the laminar boundary layer of the reactant stream into the bulk reactant stream through the reaction tubes. Convective heat transfer generally involves a thermal energy exchange between a surface and a moving fluid. The deployed wire matrix insert destroys the flow boundary layer close to the wall, and the reactant stream forms a relatively weak vortex near the wall, thereby reducing the thermal resistance of the wall fluid. The helical offset of the wire matrix insert causes the fluid to rotate, so the fluid flows from the center of the tube to the wall, and again impacts and mixes with the vortex generated by the wire loops near the wall, thereby enhancing heat transmission.

Preferably, the catalytically active wire matrix insert is adapted to enable a reactant stream flow characterized by a Reynolds number of 12000 or less, preferably 8000 or less, more preferably 2300 or less. In such low Reynolds flow regime, the pressure drop through the tube due to the wire matrix inserts is not a significant concern due to the low velocities and relatively low levels of turbulence in the flow. Due to these low Reynolds numbers, the heat transfer between the catalytically active wire matrix insert and the inner wall of the reaction tubes is better by a factor of 3 to 5 compared to a usual fixed-bed packing of individual elements, such as balls or rings. At such low Reynolds numbers, heat transport via conduction and radiation in a direction to the reaction tube walls prevails over convection. Both conduction and radiation are improved by the open wire matrix inserts. Suitably, the regions having an essentially free cross section or having wire matrix inserts having zero or limited catalytic activity, e.g. the reactant pre-heating zone, is adapted to enable a reactant stream flow characterized by a Reynolds number of 12000 or less, preferably 8000 or less.

It is, for example, possible to manipulate both the active catalyst surface and the mass of the catalyst per volume unit via the thickness of the incorporated wire. In an embodiment, the ratio of the inner diameter of the reaction tube to the diameter of the wire is in the range of about 10 to 100, preferably about 10 to 50, more preferably about 20 to 40. The wire preferably has a wire diameter ranging from 50 μm to 5000 μm, more preferably from 200 μm to 2000 μm.

Furthermore, it is possible to increase the mass transfer in the boundary layer around the catalytically active wire. The mass transfer rate in the boundary layer of such thin wires is higher compared with typically used rings or spheres due to the lower characteristic diameter of the wire used in the wire matrix inserts.

In an embodiment, the ratio of the length of the reaction zone to the length of the reactant pre-heating zone is in the range of from 0.01 to 100, preferably, 0.05 to 5, more preferably 0.1 to 1. Said ratio allows for a suitable length of the reactant pre-heating zone in the reaction tube.

In an embodiment, the reaction tubes comprise an effluent cooling zone downstream of the reaction zone. The term “effluent cooling zone” denotes a section of the reaction tube where essentially no catalytic gas-phase partial oxidation reaction occurs and where the gaseous stream through the reaction tubes is heat-exchanged via the tube wall with the circulating a heat transfer medium. This involves net heat flow out of the reaction tube. The hot effluent stream is cooled down and the heat transfer medium outside the reaction tube absorbs the heat dissipated by the effluent stream.

The effluent cooling zone preferably has an essentially free cross section or has a wire matrix insert having zero or limited catalytic activity. Preferably, the effluent cooling zone comprises a wire matrix insert. A cooling zone downstream of the reaction zone has the benefit of avoiding consecutive reactions like overoxidation to carbon oxides.

The characteristic design of a shell-and-tube heat exchange reactor is known per se to the skilled person. The shell-and-tube heat exchange reactor is limited by a “reactor shell”, which suitably is a cylindrical body, and, on the upper end and the lower end of said reactor shell, by an “upper hood” and a “lower hood”, wherein the upper hood and the lower hood are connected to the reactor shell in a gas-tight manner. Inside the shell-and-tube heat exchange reactor, a plurality of vertically arranged “reaction tubes” is present in such a way that the reactor shell encloses the plurality of reaction tubes. Generally, the term “plurality” of reaction tubes denotes a huge number of reaction tubes present inside the shell-and-tube heat exchange reactor, e.g. at least 5,000 and up to 45,000 reaction tubes. In a preferred embodiment, the reaction tubes have an inside diameter preferably in the range from 0.10 cm to 5.0 cm; more preferably in the range from 0.50 cm to 3.0 cm. Preferably, the reaction tubes have a length of at least 5 cm, preferably in the range of from 10 to 100 cm, especially 25 to 60 cm. The upper ends of the reaction tubes are connected to an “upper tube sheet” and the lower ends of the reaction tubes are connected to a “lower tube sheet”, each in a gas-tight manner. In other words, both ends of the reaction tubes are sealed in the tube sheets. Thus, a gas-tight region is formed by the space between the upper hood and the upper tube sheet, inside the reaction tubes, and the space between the lower tube sheet and the lower hood. In said region, the feed gas mixture is introduced into the shell-and-tube heat exchange reactor, subjected to a chemical reaction envisaged in the shell-and-tube heat exchange reactor inside the reaction tubes, e.g. a catalytic gas-phase partial oxidation reaction, and removed from the shell-and-tube heat exchange reactor afterwards.

During “operation mode” of the shell-and-tube heat exchange reactor, the reaction envisaged in the shell-and-tube heat exchange reactor, e.g. the catalytic gas-phase partial oxidation reaction, is performed. The reaction is performed at a reaction temperature, which is generally an elevated temperature.

Generally, in order to control the temperature of the shell-and-tube heat exchange reactor and/or to provide the elevated temperature, a heat exchange medium is circulated in a liquid-tight region between the upper tube sheet, the lower tube sheet, inside of the reactor shell and outside the reaction tubes. The heat exchange medium may, for example, be a low-melting metal such as sodium or mercury or alloys of different metals, or a liquefied salt melt of a eutectic mixture comprising nitrate moieties such as a mixture of at least two salts selected from alkali nitrates, alkali nitrites and alkali carbonates, preferably a mixture of two or three of the salts potassium nitrate, sodium nitrate and sodium nitrite.

It is very important to provide stable reaction conditions, i.e. to carry out the reaction at a constant temperature. In order to provide such stable reaction conditions, the heat transfer medium is suitably circulated through the liquid-tight region in an overall longitudinal direction at the intended temperature using a pump. Doing so enables cooling or heating of the reaction tubes, followed by cooling or heating of the heat transfer medium in a heat exchanger, e.g. an externally arranged heat exchanger. Generally, for this purpose, the reaction tubes are arranged in the shell-and-tube heat exchange reactor such that they are equidistant from each other.

Rapid cooling of the effluent stream emerging from the outlet from the reaction passage is highly desirable. This can be effected by quenching the effluent stream with an aqueous phase.

The shell-and-tube heat exchange reactor of the invention is especially, although not exclusively, suited for carrying out a catalytic gas-phase partial oxidation reaction, for example the oxidation of an alcohol to an aldehyde. Hence, the invention further relates to the use of the shell-and-tube heat exchange reactor of the invention for carrying out a catalytic gas-phase partial oxidation reaction, such as the manufacture of an aldehyde.

The invention further relates to a process for carrying out a catalytic gas-phase partial oxidation reaction comprising introducing a reactant stream into the inlet of the shell-and-tube heat exchange reactor as described above. In the process, the reactant stream comprises a partially oxidizable organic substrate and molecular oxygen, e.g. in the form of air.

In an embodiment, the precious metal is silver, the partially oxidizable organic substrate is ethylene which is catalytically oxidized to ethylene oxide.

In an embodiment, the precious metal is silver, the partially oxidizable organic substrate is methanol which is catalytically oxidized to formaldehyde.

In an embodiment, the precious metal is silver, the partially oxidizable organic substrate is an alcohol which is oxidized to an aldehyde.

A variety of ethylenically unsaturated aldehydes can be produced by the invention. In one embodiment, the ethylenically unsaturated alcohol is 3-methylbut-2-en-1-ol (prenol) and the ethylenically unsaturated aldehyde produced is prenal (3-methyl-2-buten-1-al), or the ethylenically unsaturated alcohol is 3-methylbut-3-en-1-ol (isoprenol) and the ethylenically unsaturated aldehyde produced is 3-methyl-3-buten-1-al (isoprenal). The dehydration of isoprenol to isoprenal may proceed under concomitant partial isomerization to prenal.

Generally, said process comprises the steps:
a) vaporizing the (iso)prenol;
b) admixing the (iso)prenol vapor with an oxygen-comprising gas; and
c) introducing a reactant stream into the inlet of the shell-and-tube heat exchange reactor as described above, and reacting the gaseous mixture to form (iso)prenal.

For example, in the event of the catalytic gas-phase partial oxidation reaction (iso)prenol to (iso)prenal, the reaction temperature is in the range of 300 to 500° C., preferably 350 to 400° C.

Prenol useful as a starting material for the invention may be obtained by reacting a formaldehyde source and isobutylene to obtain 3-methylbut-3-en-1-ol (isoprenol) in a reactor, in general at elevated temperature and pressure, and subjecting the obtained isoprenol to isomerization.

In one embodiment, the isoprenol is obtained by mixing and injecting a formaldehyde source and isobutylene into a reactor through at least one nozzle and reacting the formaldehyde source and isobutylene under supercritical conditions. In order to achieve supercritical conditions, formaldehyde and isobutylene are preferably reacted at a temperature of at least 220° C., for example in the range of 220 to 290° C., and an absolute pressure of at least 200 bara. The reaction of isobutene and formaldehyde may be carried out in the presence of a catalyst such as an amine base, e.g. hexamethylenetetramine (urotropin).

Herein, it is understood that any reference to “a catalyst” includes the possibilities that the catalyst is a single catalytic species or a combination of two or more different catalytic species.

Formaldehyde may be provided as a liquid, for example as a solution of paraformaldehyde. Preferably, the formaldehyde source is an aqueous formaldehyde solution. The formaldehyde source may be a single formaldehyde source or a combination of two or more different formaldehyde sources.

While initial rapid and intense mixing of reactants is desirable, it may be advantageous to continue and complete the reaction under conditions of limited back-mixing. Thus, the reaction mixture may be passed into a post-reaction chamber disposed after the reactor or in a lower portion of the reactor. In the post-reaction chamber, back-mixing is limited.

In one embodiment, the reactor comprises an upper portion and a lower portion. Injecting and mixing of the reactants occurs in a mixing chamber of the reactor disposed in the upper portion, and a fluid comprising formaldehyde and/or isobutylene and/or isoprenol is passed from the mixing chamber into a post-reaction chamber disposed in the lower portion.

Further details regarding reacting a formaldehyde source and isobutylene to obtain isoprenol may be found in WO 2020/049111 A1.

In one embodiment, reacting a formaldehyde source and isobutylene comprises mixing and injecting the formaldehyde source and isobutylene into an internal loop reactor through at least one nozzle into first conduit(s), the internal loop reactor comprising:
a vertically disposed cylindrical vessel comprising a sidewall;
at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
reactor fluid outlet means;
   wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70%, preferably at least 80%, most preferably at least 90% of the length of the draft tube; and wherein the outer surface of the draft tube(s) convexly curves so that the draft tube(s) exhibit(s) a circumferential protuberance between the tube inlet end and the tube outlet end, which circumferential protuberance is preferably located closer to the tube outlet end; wherein the convex curvature of the outer surface of the draft tube(s) extends over at least 70%, preferably at least 80%, most preferably at least 90% of the length of the draft tube; and
   wherein the edges of the draft tube(s) are rounded so that the formaldehyde source and isobutylene injected through the nozzles travel generally downward in the first conduit(s) to obtain a reacted fluid, the reacted fluid is then diverted in the opposite direction so as to travel through the second conduit and is subsequently back-mixed with the injected fluid.

This configuration of the draft tube(s) allows control of the boundary layer flowing over the edge of the draft tube. When the angle of attack of a flow with respect to a solid body reaches a certain limit, the adverse pressure gradient becomes too large for the flow to negotiate it. At this point, the flow separates from the upper surface of the body, resulting in a condition commonly known as stall. The configuration allows for decreased flow separation or a delay in flow separation, respectively. Decreased flow separation allows for reduced liquid friction and thus leads to a lower pressure drop along the streamline of the recirculating flow, which in turn results in a higher circulation ratio of the configuration. The curved shape of the inner surface of the draft tube wall guides the fluid through the draft tube in an optimized manner, comparable to the flow of fluid over an airfoil.

The inner surface of the draft tube curves in the longitudinal direction of the draft tube, or in other words has a convex shape, so that the first conduit exhibits a minimum cross-section between the tube inlet end and the tube outlet end. This means that the cross-section of the first conduit decreases from the cross-section at the tube inlet end to a minimum cross-section and increases from the minimum cross-section to the cross-section at the tube outlet end.

The draft tube has a curved, approximately conical section between the tube inlet end and the constriction, being wide at the inlet end and narrower at the constriction. At least some of the fluid flowing downstream through the draft tube is deflected so as to flow along the inner surface of the draft tube until the draft tube ends. The flow through the tube predominantly remains attached, thus generating less pressure loss. In the vicinity of the constriction, the fluid flowing downstream through the draft tube is accelerated. Between the constriction and the tube outlet end, the cross-sectional area of the draft tube widens again. Consequently, the area variation, in conjunction with mass conservation, will force the velocity through the larger area to be slower than through the smaller one, accompanied by a conversion of the dynamic pressure into static pressure. Acceleration of the fluid flowing downstream through the draft tube in the vicinity of the constriction adds a radial velocity component to the flow, increasing the mixing between circulating flow and injected flow. By avoiding flow separation in this case, no significant pressure loss occurs.

In a preferred embodiment, the nozzles are two-component nozzles. It is especially preferable that a two-component nozzle is designed so as to provide an annular jet of isobutylene around a central jet of the formaldehyde source, and that the injection velocities of these two jets are different. In this embodiment, the jet of isobutylene has a large shear surface towards both the central jet of the formaldehyde source and the reaction mixture in the reactor, allowing for favorable fast mixing of the reactants.

In a preferred embodiment, the loop reactor comprises deflector means arranged between the nozzle and the draft tube, the deflector means being suitable for deflecting fluid travelling in the second conduit in the opposite direction.

The deflector means suitably comprise a surface which is concave relative to the end of the draft tube which defines the tube inlet end. In a preferred embodiment, the deflector means have a partial toroidal surface. It is especially preferred that the deflector means are provided in the shape of the upper portion of a ring torus bisected in a plane parallel to the toroidal direction. This shape allows for an especially efficient deflection of the fluid travelling in the second conduit. The deflector means may allow for a stabilization of the injected fluid stream. This is especially relevant when the flow rate of the fluid travelling in the second conduit is not uniform across the cross section of the reactor, which may lead to an eccentricity of the injected fluid stream. Such an eccentricity may cause a decrease in circulation ratio if left unattended.

When the first conduit is downcomer conduit and the second conduit is a riser conduit, it is preferred that the shape of the deflector means constitutes the upper portion of a ring torus bisected in a plane parallel to the toroidal direction, wherein the ring torus is bisected at at least 50% of its height, such as at least 55% or 65% of its height. Thus, the upper portion of the ring torus is the same size or smaller than the lower portion of the ring torus. In another preferred embodiment, the shape of the deflector means constitutes the upper portion of a ring torus bisected in a plane parallel to the toroidal direction wherein the ring torus is bisected at at most 85% of its height, for example 80% of its height. In these ranges, the entry of the deflector means is angled especially suitable for fluid deflection.

High temperatures are required to obtain a high isoprenol yield in the reaction of formaldehyde with isobutylene. Effective removal of the heat is critical for the product quality and process safety. The heat removed from the isoprenol is used for raising the temperature of isobutylene before it enters the reactor. The stream of the hot isoprenol contains sensible heat from the chemical reaction. The sensible heat is potentially reclaimable energy that can be reused.

Advantageously, reacting a formaldehyde source and isobutylene preferably comprises heat-exchanging a stream of hot isoprenol withdrawn from the reactor with a isobutylene stream directed to the reactor; wherein heat-exchanging is performed in at least two shell-and-tube heat exchangers; each of the heat exchangers comprising a plurality of tubes and a shell-side heat exchange passage; wherein the hot isoprenol is directed through the tubes of the heat exchangers; and the isobutylene is guided through the shell-side passage, and at least two of the heat exchangers are connected in series with regard to both the shell-side flow and the tube-side flow.

Such configurations allow for prolonging operation intervals between maintenance disruptions in such a method. The term “maintenance disruptions” is intended to mean a shutdown of the process that becomes necessary at recurring intervals in order to clear the tubes of the heat exchanger that have been clogged by fouling. An indicator of a necessity of a maintenance disruption is typically when isobutylene leaving the last heat exchanger is insufficiently pre-heated and that even a subsequent heater is hardly able to put in additional external heat into the isobutylene to bring isobutylene to the required temperature before it enters the reactor. One aspect of the invention is that the pre-heating of the isobutylene stream can be maintained for a longer time at levels high enough so that the desired temperature of the isobutylene can easily be reached before the isobutylene enters the reactor.

One particular area prone to fouling in conventional shell-and-tube heat exchangers is the tube area near the tube sheet near the inlet where the tube-side fluid leaves the individual tubes. Excessive fouling in this area can cause clogging of individual tubes and fluid stagnation along the entire length of these tubes. The fluid stagnation generally leads to reduced heat-transfer performance.

As a further consequence of the decreased heat transfer performance caused by fouling, the energy required in a heater to adjust the temperature of the pre-heated isobutylene stream to the desired reaction temperature increases. Consequently, more additional external heat becomes necessary which is detrimental in terms of energy demand and process economy, and often has a negative impact on the carbon dioxide footprint of the product.

By using two or more heat exchangers, the impact of fouling in individual tubes on the overall heat exchange capacity is reduced in comparison to arrangements where only a single heat exchanger is used. As a consequence, the heat transfer rates are maintained at a desired level for longer periods, hence prolonging operation intervals between maintenance disruptions, and the pre-heating of the isobutylene stream requires less additional external heat compared to a plant with a single heat exchanger in an advanced state of fouling.

Separation of the isoprenol from unreacted formaldehyde is not a trivial task. This difficulty arises from the fact that monomeric formaldehyde (as well as polymeric formaldehyde) forms both hydrates with water and hemiformals with isoprenol. The hydrates and hemiformals of varying formaldehyde polymerization degree have intermingling boiling points.

It has, however, been found that formaldehyde can be separated virtually completely from isoprenol via distillation at a temperature at which the hemiformal is cleaved to formaldehyde and isoprenol, so that the formaldehyde can be easily separated from the isoprenol.

Hence, crude isoprenol may be purified by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

In particular, it has been found that the formaldehyde can be separated virtually completely from isoprenol and concentrated aqueous formaldehyde suitable for recycling into the isoprenol synthesis can be obtained in a distillation train involving a first distillation at a temperature at which the equilibrium is shifted towards the hemiformal of formaldehyde and isoprenol, so that essentially all formaldehyde remains in the bottoms of the distillation, and a second distillation at a temperature at which the hemiformal is cleaved to formaldehyde and isoprenol, so that the formaldehyde can be easily separated from the isoprenol.

In order to permit a first distillation at a temperature below the isoprenol-formaldehyde dissociation temperature and a second distillation at a temperature above the isoprenol-formaldehyde dissociation temperature, two low-boiler separation towers operated at different pressures are envisioned. Hence, at the relatively low pressure prevailing in the first low-boiler separation tower, a first distillate containing water and low-boilers essentially free of formaldehyde is obtained. At the relatively high pressure prevailing in the second low-boiler separation tower, a virtually all formaldehyde is separated from the isoprenol. This process thus allows for obtaining isoprenol essentially free of formaldehyde.

Hence, in a more preferred embodiment, the purification process comprises
(i) directing the stream of crude isoprenol to a first low-boiler separation tower operated at a pressure of 1.5 bara or lower, to obtain a first bottoms stream containing isoprenol and formaldehyde, and a first distillate stream containing water and low-boilers;
(ii) directing the first bottoms stream to a second low-boiler separation tower operated at a pressure of 2 bara or higher, to obtain a second distillate stream containing aqueous formaldehyde, and a second bottoms stream containing isoprenol; and
(iii) directing the second bottoms stream to a finishing tower to obtain pure isoprenol as a distillate stream, and a bottoms stream containing high-boilers.

The second distillate stream constitutes concentrated aqueous formaldehyde fit for recycle into the isoprenol synthesis.

Suitably, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200° C., more preferably 170 to 185° C., most preferably 175 to 180° C. The temperature at the top of the second low-boiler separation tower is preferably in the range of 115 to 160° C., more preferably 125 to 145° C.

In a particularly preferred embodiment, the second low-boiler separation tower is operated at a pressure in the range of 2.9 to 3.5 bara, a bottoms temperature in the range of 175 to 180° C. and a temperature at the top in the range of 130 to 140° C.

Further information on the process for recovering isoprenol essentially free of formaldehyde may be found in WO 2022/189652 A1.

The obtained isoprenol may be subjected to catalytic isomerization so as to obtain prenol.

Isomerization of isoprenol to 3-methyl-2-buten-1-ol (prenol) may be carried out over a supported noble metal, preferably in the presence of hydrogen. A preferred catalyst is a fixed bed catalyst containing palladium and selenium or tellurium or a mixture of selenium and tellurium supported on silicium dioxide. The isomerization is carried out at a temperature of 50 to 150° C. to produce a reaction mixture of prenol and isoprenol. The isoprenol can be recycled. Further details are provided in WO 2008/037693.

The (iso)prenol obtained as described above may be subjected to the catalytic gas-phase partial oxidation reaction of the invention. Prior to contacting with the catalytically active wire matrix insert, the (iso)prenol may advantageously be treated to remove organically bound nitrogen from the (iso)prenol by contacting the (iso)prenol with a weakly acidic solid adsorbent. In other words, the (iso)prenol may be depleted of organically bound nitrogen by this process.

The term “organically bound nitrogen” is intended to denote any compound containing at least one nitrogen atom directly bound to one or more carbon atoms. For example, such compounds containing at least one nitrogen atom may be selected from amines, such as ethylamine, trimethylamine, aniline, pyridine or piperidine. An amine particularly significant in practice is hexamethylenetetramine (urotropin). (Iso)prenol may comprise about 5 to 30 ppm of organically bound nitrogen.

The weakly acidic solid adsorbents have been found to be capable of adsorbing organically bound nitrogen in the presence of abundant (iso)prenol while not interfering with the reactive carbon-carbon double bond.

The weakly acidic adsorbent may include an adsorbent material having sufficient acidity to adsorb the organically bound nitrogen from the (iso)prenol. In an embodiment, the solid adsorbent is a crosslinked resin having phosphonic functional groups. Preferably, the resin polymer is a vinyl aromatic copolymer, preferably crosslinked polystyrene and more preferably a polystyrene divinylbenzene copolymer. Other polymers having a phosphonic functional group may also be used. Preferably, the crosslinked resin having phosphonic functional groups is of the macroporous type. A preferred solid adsorbent is Purolite S956.

The resin is typically used in bead form and loaded into a column. The (iso)prenol is passed through the column, contacting the resin beads. During contact, the organically bound nitrogen in the (iso)prenol reacts with the functional group and an exchange occurs where a proton is transferred to the nitrogen and an ionic bond is formed to the anionic site of the resin. Contact is maintained until a threshold level is reached i.e. the breakthrough concentration. At this breakthrough point, the process reaches an equilibrium where additional organically bound nitrogen cannot be removed effectively. The flow is halted and the column is backwashed with water, preferably deionized or softened water. By flowing in reverse, the resin is fluidized and solids captured by the beads are loosened and removed.

In another embodiment, the solid adsorbent is a silica-alumina hydrate. Numerous silica-alumina catalyst compositions and processes for their preparation are described in the patent literature, see, e.g., U.S. Pat. No. 4,499,197.

Preferably, the alumina content of the silica-alumina hydrate is from about 10 to about 90 wt.-% of Al₂O₃. The preferred range of alumina content is from about 30 to about 70 wt.-% of Al₂O₃.

The introduction of silicon dioxide into aluminum oxide leads to the introduction of acidic centers. The number of acidic centers can be controlled by the amount of introduced silicon dioxide. The number of acidic centers increases with the amount of introduced silicon dioxide up to a maximum number of acidic centers, and decreases again with a further increasing amount of silicon dioxide after having reached the maximum number of acidic centers.

Examples of commercially available silica-alumina hydrates are Siral® available from Sasol Germany Gmbh, Hamburg, Germany. Siral® is based on orthorhombic aluminum oxide hydroxide (boehmite; AlOOH) and doped with SiO₂. Various Siral® grades having different ratios of Al₂O₃ to SiO₂ are available: Siral 1 (Al₂O₃/SiO₂=99/1), Siral 5 (Al₂O₃/SiO₂=95/5), Siral 10 (Al₂O₃/SiO₂=90/10), Siral 20 (Al₂O₃/SiO₂=80/20), Siral 28M (Al₂O₃/SiO₂=72/28), Siral 30 (Al₂O₃/SiO₂=70/30), Siral 40 (Al₂O₃/SiO₂=60/40). Siral 40 is especially preferred.

In an embodiment, the (iso)prenol is passed over a bed of the weakly acidic solid adsorbent. Suitably, said step of “passing over a bed” denotes that a layer (“bed”) of the weakly acidic solid adsorbent is provided in a customary reaction vessel known to the skilled person which may preferably be equipped with a stirring device, e.g. in a stirred-tank reactor. The (iso)prenol is then introduced into the reaction vessel and guided through the same in a manner that it gets into contact with the weakly acidic solid adsorbent.

Alternatively, the weakly acidic solid adsorbent may be provided in a reaction tube, e.g. of a tubular reactor and the (iso)prenol then continuously flows through said reaction tube(s) while getting into contact with the weakly acidic solid adsorbent.

In an embodiment, the (iso)prenol comprises, after contacting the alcohol stream with a weakly acidic solid adsorbent, less than 2 ppm of organically bound nitrogen. Herein, “ppm” denotes wt.-ppm of compounds incorporating organically bound nitrogen, relative to the total weight of the (iso)prenol.

Suitably, the content of organically bound nitrogen in the (iso)prenol may be determined by Kjeldahl analysis. Alternatively, an oxidative combustion method with a chemiluminescence detector according to DIN 51444 may be used.

When isoprenol is subjected to the catalytic gas-phase partial oxidation reaction of the invention, it may be favorable to maintain in the reactant stream a weight ratio of formaldehyde to isoprenol of less than 0.04, preferably less than 0.03, in particular less than 0.02, or less than 0.01. In still more preferred embodiments, the weight ratio of formaldehyde to isoprenol is maintained at less than 0.002, or less than 0.001.

The weight ratio of formaldehyde to isoprenol in the reactant stream may be maintained at a certain level or less. Reducing the weight ratio of formaldehyde to isoprenol in the reactant stream beyond a certain point, however, reaches a point of rapidly diminishing return. Formaldehyde removal involves additional equipment and operating costs. An economic balance must be taken between the improvement due to reducing the ratio and the cost of maintaining such a ratio. Hence, the weight ratio of formaldehyde to isoprenol is preferably not lower than 0.0005 or, in some instances, not lower than 0.005.

It has been found that reactor clogging and pressure drop increase are significantly affected by the presence of formaldehyde in the reactant stream. Catalyst-fouling reactions of condensation and polymerization are believed to be the principal reactions involved in carbon or coke formation on the catalyst. It is thought that this carbon formation involves thermal condensation of formaldehyde or of formaldehyde with the olefinic hydrocarbons isoprenol and (iso)prenal. In the presence of the catalyst, the primary condensation products tend to undergo dehydrogenation and polymerization type reactions and to settle on the catalyst and undergo further dehydrogenation and decomposition until carbonaceous deposits are formed.

The presence of formaldehyde in the reactant stream is due to two main sources. Formaldehyde may be contained in the fresh feed stream sent to the reactor, that is as an impurity originating from the isoprenol manufacture step. All the formaldehyde that cannot be separated in the purification step following the isoprenol synthesis ends up in the reactant stream.

In addition, formaldehyde is also generated in situ. Part of the isoprenol splits back to isobutene and formaldehyde. Since most continuous industrial processes operate at single-pass conversion levels of 50 to 60% and with recycling of the unconverted isoprenol, formaldehyde may be present in the recycling stream of unconverted isoprenol, if no steps to purify the stream containing unreacted isoprenol are taken. The recycle stream of unconverted isoprenol has now been found to constitute the biggest source of formaldehyde contamination in the reactant stream. The process is generally carried out at partial conversions, for example at conversions of 30 to 70%, preferably 50 to 60%. An unreacted isoprenol stream is separated from the product stream. The unreacted isoprenol stream is recycled, that is, combined with a fresh feed stream comprising isoprenol to provide the reactant stream. The unreacted isoprenol stream comprises isoprenol as a main constituent, but may also comprise prenal, isoprenal, isoamylalcohol, isovaleraldehyde, isovaleric acid, prenol, formaldehyde. It can also contain traces of other C₃ and C₂ aldehydes and acids.

According to the invention, reducing the weight ratio of formaldehyde to isoprenol in the reactant stream can be accomplished in several different ways. In an embodiment, formaldehyde is removed from the unreacted isoprenol stream prior to combining the unreacted isoprenol stream with the fresh feed stream.

In an embodiment, the unreacted isoprenol stream is combined with the fresh feed stream and formaldehyde is removed from the combined stream.

Alternatively, it is feasible to mix the unreacted isoprenol stream with an amount of a sufficiently purified fresh feed stream so as to give in the combined stream a desired weight ratio of formaldehyde to isoprenol.

In an embodiment, the fresh feed stream comprising isoprenol is derived from a process reacting iso-butene and formaldehyde and is purified to a weight ratio of formaldehyde to isoprenol of less than 0.04, preferably less than 0.03, in particular less than 0.02, or less than 0.01. In still more preferred embodiments, the fresh feed stream is purified to weight ratio of formaldehyde to isoprenol of less than 0.002, or less than 0.001.

Formaldehyde may be removed from isoprenol streams by a conventional separating method such as distillation, selective adsorption and or selective reaction, in particular by the purification process involving the pressure-swing distillation as described above.

The prenal produced by the invention is a useful intermediate in the preparation of citral. Citral is a mixture of the isomeric compounds neral and geranial.

3,7-dimethyl-octa-2,6-dienal (citral) can be prepared by obtaining prenal by a process as described above, further comprising the steps of condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

In particular, 3,7-dimethyl-octa-2,6-dienal (citral) can be prepared by a process comprising the steps of:
a) condensing the prenal with prenol in the presence of at least one catalyst in a reaction column while withdrawing an acetal fraction comprising the diprenyl acetal of prenal from the reaction column;
b) subjecting the acetal fraction in a cleaving column to cleaving conditions in the presence of at least one catalyst while withdrawing from the cleaving column a cleaving fraction containing at least one of prenyl (3-methyl-butadienyl) ether and 2,4,4-trimethyl-3-formyl-1,5-hexadiene, and optionally containing citral; and
c) reacting the cleaving fraction in a plug-flow type reactor to obtain citral.

The overall reaction sequence is illustrated by the reaction scheme below.

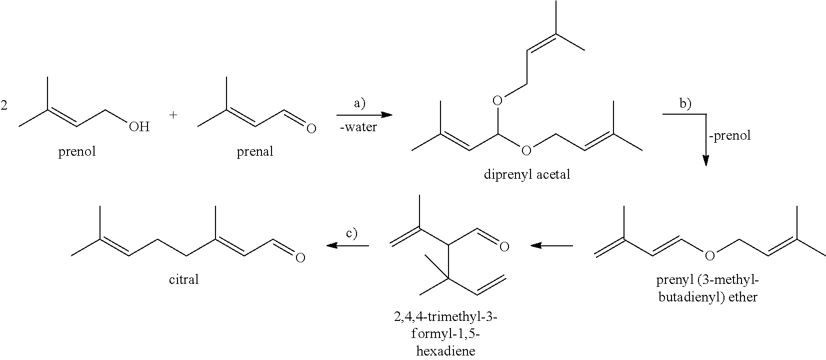

In step a), the unsaturated acetal 3-methyl-2-butenal-diprenyl acetal (herein referred to as “diprenyl acetal of prenal” or “diprenyl acetal”) is formed from prenol and prenal using a catalyst. For this purpose, prenal is reacted together with prenol in the presence of catalytic amounts of an acid and with separation of the water formed during the reaction in a reaction column. In step b), the resulting 3-methyl-2-butenal diprenyl acetal (diprenyl acetal) of step a) is cleaved in the presence of a catalyst in a cleaving column with elimination of 3-methyl-2-buten-1-ol (prenol) to give prenyl (3-methylbutadienyl) ether. Claisen rearrangement of the obtained prenyl (3-methylbutadienyl) ether yields 2,4,4-trimethyl-3-formyl-1,5-hexadiene which subsequently undergoes Cope rearrangement yielding 3,7-dimethyl-2,6-octadienal (citral).

Step a) is carried out in the presence of a catalyst, preferably an acid. In an embodiment, the catalyst in step a) is nitric acid.

Preferably, in steb b), the acetal fraction is continuously subjected to cleaving conditions in a cleaving column. “Cleaving conditions” denotes reaction conditions selected such that the diprenyl acetal contained in the acetal fraction is cleaved to prenyl (3-methylbutadienyl) ether which may subsequently rearrange to 2,4,4-trimethyl-3-formyl-1,5-hexadiene and citral.

The acetal fraction comprises diprenyl acetal as a main constituent. The acetal fraction does not necessarily need to consist of pure diprenyl acetal, but may also comprise prenol, prenal and citral building blocks.

Step b) is carried out in the presence of a catalyst, preferably an acid catalyst. Suitable acid catalysts are selected from non-volatile protic acids such as sulfuric acid, p-toluenesulfonic acid and phosphoric acid.

Suitably, the continuous cleaving in the cleaving column of step b) may be carried out in the lower part or the sump of the distillation column acting as cleaving column. Preferably, the acetal fraction and/or the catalyst(s) are introduced into the lower part of the distillation column, into the sump of the distillation column or into the evaporator of the distillation column.

If desired, a high-boiling inert compound can be introduced into the sump of the cleaving column in order to ensure a minimum filling level of the sump and the evaporator. Suitable high-boiling inert compounds are selected from liquid compounds which are inert under the reaction conditions and have a higher boiling point than citral and diprenyl acetal. For example, the high-boiling inert compounds may be selected from hydrocarbons such as tetradecane, pentadecane, hexadecane, octadecane, eicosane; or ethers such as diethylene glycol dibutyl ether; white oils; kerosene oils; or mixtures thereof.

Suitably, the distillation conditions are selected such that the diprenyl acetal is predominantly retained in the lower part or the sump of the distillation column. During the cleaving reaction, a cleaving fraction is continuously withdrawn from the cleaving column, the cleaving fraction containing at least one of prenyl (3-methyl-butadienyl) ether and 2,4,4-trimethyl-3-formyl-1,5-hexadiene, and optionally containing citral. For the ease of reference, prenyl (3-methyl-butadienyl) ether, 2,4,4-trimethyl-3-formyl-1,5-hexadiene and citral are collectively referred to as “citral building blocks”. This is because the former are intermediates on the reaction route to citral and can be converted into citral in the subsequent step c).

Additionally, the prenol formed during the cleaving reaction in step b) is continuously removed from the reaction mixture, generally at the top of the cleaving column.

The cleaving fraction together with the formed prenol may be withdrawn at the top of the distillation column.

Alternatively and preferably, it is also possible to withdraw the cleaving fraction in liquid or vaporous form at a side draw of the distillation column.

In step c), the cleaving fraction is reacted in a plug-flow type reactor to obtain citral. To this end, the cleaving fraction is guided through the plug-flow type reactor at a suitable temperature for carrying out the rearrangement reaction(s) yielding citral. By employing a combination of a highly back-mixed cleaving column and a plug-flow reactor, it is possible to increase the selectivity and the yield of the cleaving reaction. All of the catalyst(s) required for the cleaving reaction is preferably introduced into the cleaving column in step b) and preferably, no catalyst is introduced into the plug-flow reactor.

In an embodiment, prenol eliminated in step b) is recycled to step a). This allows for improved yields to be achieved in the process of the invention.

In one aspect, the invention hence relates to an improved process for the preparation of citral (3,7-dimethyl-octa-2,6-dienal), comprising the steps of
A) reacting a formaldehyde source and isobutylene to obtain 3-methylbut-3-en-1-ol (isoprenol), and subjecting at least part of the obtained isoprenol to isomerization to obtain prenol;
B) preparing prenal (3-methylbut-2-en-1-al) from 3-methylbut-2-en-1-ol (prenol) using the processes described above and/or preparing prenal using the processes described above via isoprenal (3-methylbut-3-en-1-al) made from isoprenol; and
C) condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

Step A can be performed as described above or by other methods known in the art, preferably via distillation at a temperature at which the hemiformal is cleaved to formaldehyde and isoprenol, so that the formaldehyde can be easily separated from the isoprenol, and more preferably by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

Step B comprises oxidative dehydrogenation of prenol and/or isoprenol. The conversion of isoprenol with a catalytically active metal catalyst forms a reaction mixture of 3-methylbut-3-en-1-al and 3-methylbut-2-en-1-al. The former isomer may then isomerize under base catalysis to give the desired 3-methylbut-2-en-1-al.

Step C can be performed as described above, for example via steps a) to c).

The thus obtained citral is a useful intermediate for, e.g., menthol or linalool.

Menthol may be prepared from citral via a process comprising the steps of
catalytic hydrogenation of citral to obtain citronellal;
cyclization of citronellal to obtain isopulegol in the presence of an acidic catalyst; and
catalytic hydrogenation of isopulegol to obtain menthol.

The overall reaction sequence is illustrated by the reaction scheme below.

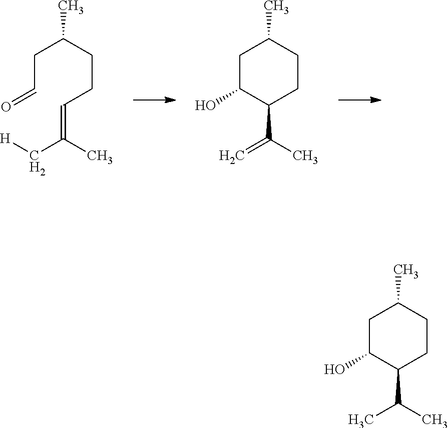

The hydrogenation of citral to obtain citronellal may be achieved by hydrogenation in the presence of a rhodium-phosphine catalyst.

The cyclization of citronellal to isopulegol may be achieved by cyclization in the presence of a Lewis-acidic aluminum-containing catalyst, such as a bis(diarylphenoxy)aluminum compound, which may be used in the presence of an auxiliary, such as a carboxylic anhydride. The isopulegol may be recovered from the catalyst containing reaction product by distillative separation to give an isopulegolenriched top product and an isopulegol-depleted bottom product. From the bottom product, the catalyst may be regenerated. The isopulegol obtainable in this way by the cyclization of citronellal can be further purified by suitable separating and/or purification methods, in particular by crystallization, and be at least largely freed from undesired impurities or by-products.

The hydrogenation of isopulegol may be achieved by hydrogenation in the presence of a heterogeneous nickel-containing catalyst, preferably a heterogeneous nickel and copper-containing catalyst.

Further details regarding the reaction sequence from citral to menthol may be found in US 2013/46118 A1, which is incorporated by reference herein.

In one aspect, the invention thus relates to an improved process for the preparation of menthol by producing citral using the above processes and then producing menthol from the citral. Menthol may be prepared as described herein or by other methods known in the art.

Linalool may be prepared from citral via a process comprising catalytic hydrogenation of citral to obtain nerol and/or geraniol, and isomerization thereof.

The hydrogenation of citral to obtain nerol and/or geraniol may be achieved by hydrogenation in the presence of a supported ruthenium, rhodium, osmium, iridium or platinum catalyst, preferably a ruthenium catalyst supported on carbon black.

The isomerization of nerol and/or geraniol to obtain linalool may be achieved by isomerization in the presence of a tungsten catalyst, in particular a dioxotungsten (VI) complex. Further details regarding the isomerization of nerol and/or geraniol may be found in U.S. Pat. No. 7,126,033 B2.

In one aspect, the invention thus relates to an improved process for the preparation of linalool by producing citral using the above processes and then producing linalool from the citral. Linalool may be prepared as described herein or by other methods known in the art.

The invention is further illustrated by the examples and FIGURE that follow. However, it will be understood that the examples and FIGURE are not intended to limit the scope of the invention in any wax.

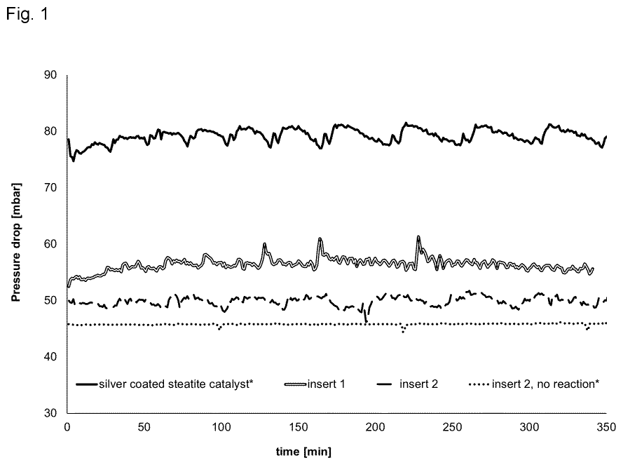

FIG. 1 depicts a diagram of the pressure drop over time for three different catalytically active structures.

### EXAMPLES

Prenol was continuously vaporized in a double-pipe vaporizer. The prenol vapor was introduced into a reaction tube at the bottom of said reaction tube at a flow rate of 300 g/h, a temperature of 365° C. and a pressure of 1 atm. Together with the prenol vapor, air was introduced into the reaction tube at the bottom of the reaction tube at a flow rate of 100 NI/h. The reaction tube had an inner diameter of 12 mm and a length of 500 mm. An effluent stream was recovered at the top of the reaction tube and analyzed by gas chromatography.

Different catalytically active structures were tested in the reaction tube:
a wire matrix insert (available from Calgavin) manufactured of massive silver wire having a void fraction of 90.6% and a length of 300 mm (insert 1),
a wire matrix insert (available from Calgavin) manufactured of massive silver wire having a void fraction of 90.6% and a length of 150 mm (insert 2), or
a packing of silver coated steatite catalysts having a diameter of 2 mm.

The wire matrix inserts were placed into the reaction tube such that one end of the wire matrix insert was located at the outlet of the reaction tube, i.e. resulting in a reactant pre-heating zone having a length of 200 mm (with insert 1) or 350 mm (insert 2). The silver coated steatite catalyst was placed at the outlet of the reaction tube such that it filled 300 mm of the reaction tube, i.e. resulting in a pre-heating zone having a length of 200 mm.

Three runs for each insert 1 and insert 2 (inventive examples) as well as for the silver coated steatite catalyst (comparative example) have been carried out. The results are shown in tables 1 to 3 and FIG. 1.

The normalized selectivities (selectivity of the silver coated steatite catalyst=100%) are shown in table 1.

TABLE 1

| catalyst | normalized selectivity [%] |
|---|---|
| silver coated | 100 |
| steatite catalyst* | 100 |
| | 100 |
| insert 1 | 92 |
| | 85 |
| | 87 |
| insert 2 | 107 |
| | 123 |
| | 116 |

*comparative example

The conversion of prenol and selectivity of prenal is shown in table 2.

TABLE 2

| catalyst | conversion [%] | selectivity [%] |
|---|---|---|
| silver coated | 64.6 | 78.1 |
| steatite catalyst* | 63.9 | 71.2 |
| | 65.8 | 80.8 |
| insert 1 | 65.4 | 70.2 |
| | 62.9 | 65.3 |
| | 61.8 | 66.8 |
| insert 2 | 61.5 | 82.4 |
| | 60.2 | 94.5 |
| | 57.7 | 89.0 |

*comparative example

The amounts of gaseous products formed and average selectivities of prenal are shown in table 3.

TABLE 3

| | liquids mass loss |
|---|---|
| catalyst | [g/h] ^{[1]} |
| silver coated | 53 |
| steatite catalyst* | |
| insert 1 | 60 |
| insert 2 | 48 |

^{[1]} calculated as flow rate of liquid feed in g/h minus flow rate of liquid product in g/h
*comparative example

Liquids mass loss is a measure for side reactions leading to gaseous products such as overoxidation to CO and/or CO₂.

FIG. 1 depicts the pressure drop vs. reaction time for three different catalytically active structures: insert 1 and insert 2 (inventive examples), and silver coated steatite catalyst (comparative example). For reference purposes, a run with insert 2 at 350° C. is also included in FIG. 1. At 350° C., no reaction occurs and therefore, this run exemplifies the lowest possible pressure drop with no depositions or coke formation at all.

It can be seen from FIG. 1 that the pressure drop of the examples according to the invention was lower (almost reduced by factor 2) than the pressure drop upon using the silver coated steatite catalyst (comparative example). This advantageously leads to a longer time-on-stream and less shut-down time per year.

1.-20. (canceled)

21. A shell-and-tube heat exchange reactor for carrying out a catalytic gas-phase partial oxidation reaction comprising
a shell-side heat exchange passage for circulating a heat transfer medium and a reaction passage comprising a plurality of reaction tubes;
an inlet for introducing the reactant stream to the reaction passage; and
an outlet from the reaction passage for recovering an effluent stream from the reaction tubes;
wherein the reaction tubes comprise
a reactant pre-heating zone adjacent to the inlet, and
a reaction zone downstream of the reactant pre-heating zone, the reaction zone having a catalytically active wire matrix insert having at least on a part of its surface a catalytically active precious metal.

22. The shell-and-tube heat exchange reactor of claim 21, wherein the reactant pre-heating zone has an essentially free cross section or has a wire matrix insert having zero or limited catalytic activity.

23. The shell-and-tube heat exchange reactor of claim 21, wherein the ratio of the length of the reaction zone to the length of the reactant pre-heating zone is in the range of from 0.01 to 100.

24. The shell-and-tube heat exchange reactor of claim 21, wherein the reaction zone comprises an alternating series of regions having catalytically active wire matrix inserts and regions having an essentially free cross section or having wire matrix inserts having zero or limited catalytic activity.

25. The shell-and-tube heat exchange reactor of claim 21, wherein the reaction tubes comprise an effluent cooling zone downstream of the reaction zone, wherein the effluent cooling zone has an essentially free cross section or has a wire matrix insert having zero or limited catalytic activity.

26. The shell-and-tube heat exchange reactor of claim 21, wherein the catalytically active precious metal is selected from copper, silver, palladium, platinum, ruthenium, and rhodium.

27. The shell-and-tube heat exchange reactor of claim 22, wherein the wire matrix insert having zero or limited catalytic activity is made of an inert material.

28. The shell-and-tube heat exchange reactor of claim 21, wherein the catalytically active wire matrix inserts comprise an elongated core having a plurality of wire loops extending from the elongated core, wherein the wire loops are longitudinally arranged and helically shifted, and wherein the wire loops comprise a massive precious metal wire, or a wire coated with a precious metal.

29. The shell-and-tube heat exchange reactor of claim 28, wherein the elongated core comprises at least two longitudinal core wire members, which are twisted around each other to form core wire windings, and the wire loops are accommodated in the core wire windings.

30. The shell-and-tube heat exchange reactor of claim 28, wherein the ratio of the inner diameter of the reaction tube to the diameter of the massive precious metal wire or the wire coated with a precious metal is in the range of about 10 to 100.

31. The shell-and-tube heat exchange reactor of claim 21, wherein the reaction zone has a void fraction of 0.60 to 0.99.

32. The shell-and-tube heat exchange reactor of claim 21, wherein the catalytically active wire matrix insert is adapted to enable radial mixing of the laminar boundary layer of the reactant stream into the bulk reactant stream through the reaction tubes.

33. A process for carrying out a catalytic gas-phase partial oxidation reaction, the process comprising: introducing a reactant stream into the inlet of the shell-and-tube heat exchange reactor of claim 21, wherein the reactant stream comprises a partially oxidizable organic substrate and molecular oxygen.

34. The process according to claim 33, wherein the flow of the reactant stream inside the pre-heating zone is essentially laminar.

35. The process according to claim 33, wherein the flow of the reactant stream inside the reaction zone containing the catalytically active wire matrix insert is characterized by a Reynolds number of 12000 or less.

36. The process according to claim 33 for the manufacture of an aldehyde, wherein the precious metal is silver and the partially oxidizable organic substrate is an alcohol.

37. The process according to claim 36, wherein the alcohol is isoprenol, and wherein the isoprenol is obtained by reacting at least one formaldehyde source and isobutylene in a reactor to obtain isoprenol.

38. The process according to claim 36, wherein the partially oxidizable organic substrate is isoprenol, wherein the process additionally comprises at least one of αα, ββ and γγ:
αα) purifying of isoprenol by subjecting a stream of crude isoprenol containing isoprenol, water and formaldehyde, or an isoprenol containing fraction thereof, to distillation in a low-boiler separation tower operated at a pressure of 2 bara or higher, to obtain a distillate stream containing aqueous formaldehyde and a bottoms stream containing isoprenol essentially free of formaldehyde;
ββ) maintaining in the reactant stream a weight ratio of formaldehyde to isoprenol of less than 0.04;
γγ) treating the (iso)prenol to remove organically bound nitrogen from the (iso)prenol by contacting the isoprenol with a weakly acidic solid adsorbent prior to contacting with the catalytically active wire matrix insert.

39. A process for the preparation of 3,7-dimethyl-octa-2,6-dienal (citral), the process comprising: obtaining prenal by the process according to claim 36, further comprising the steps of condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

40. A process for the preparation of a citral-derived chemical, comprising preparing citral by the process according to claim 39, and at least one of ααα, βββ or (βββ plus γγγ):
ααα) converting the citral to obtain menthol;
βββ) converting the citral to geraniol and/or nerol;
γγγ) converting the geraniol and/or nerol to obtain linalool.
